(19) United States
(12) Patent Application Publication
(10) Pub. No.: US 2010/0228214 A1
(43) Pub. Date: Sep. 9, 2010

(54) Polypropylene mixture

(75) Inventors: Steffen Bornemann, Jessnitz (DE); Markus Haberer, Osnabruck (DE); Helmut Hartl, Braunschweig (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Fiberweb Corovin GmbH

(21) Appl. No.: 12/712,699

(22) Filed: Feb. 25, 2010

Related U.S. Application Data
(63) Continuation of application No. PCT/EP2008/008568, filed on Oct. 10, 2008.

(30) Foreign Application Priority Data
Oct. 11, 2007 (DE) 10 2007 049 031.5

(51) Int. Cl. A61F 13/514; D01F 8/06; D04H 3/16; C08L 23/12

(52) U.S. Cl. 604/372; 442/364; 442/401; 428/219; 156/167; 428/315.5; 525/240

(57) ABSTRACT

The present invention relates to the use of a polypropylene mixture for the production of a spunbonded non-woven fabric having increased elastic property, wherein the polypropylene mixture substantially comprises a first homopolypropylene and a second homopolypropylene, wherein an MFR of the first homopolypropylene is greater than an MFR of the second homopolypropylene, wherein the second homopolypropylene has a weight proportion in the polypropylene mixture of at least 3% by weight to a maximum of 25% by weight, wherein the first homopolypropylene is substantially the remaining weight proportion of the polypropylene mixture, wherein the second homopolypropylene has an MFR between 0.7 and 14 g/10 min (230° C./2.16 kg) according to ISO 1133, and a difference of the MFR of the second homopolypropylene to the MFR of the first homopolypropylene is at least 10 g/10 min, and an upper limit of the MFR of the first homopolypropylene is 55 g/10 min (230° C./2.16 kg) according to ISO 1133. Substantially here shall mean that only the two homopolypropylenes are used as polymers. Additionally, additives can be added, but no further polymer. The invention further relates to a corresponding polypropylene mixture, a spunbonded non-woven fabric made from said polypropylene mixture, and a corresponding method of production.

The present invention concerns the use of a polypropylene mixture to produce a spunbond nonwoven with increased elongation property, the corresponding polypropylene mixture, a spunbond nonwoven produced with such a polypropylene mixture and a method for production of a spunbond nonwoven with increased elongation property.

Mixtures of different polymers, often also called blends, are produced especially from polyols to product spunbond nonwovens. These include polypropylene and polyethylene, which are mixed with each other. Blends of PP copolymers with polyethylene or ethylene copolymers follow from WO 01/73174. If one intends to increase the stretchability of the spunbond nonwoven fiber, a thermoplastic-elastomer polyolefin is often used instead of polypropylene. This is apparent for example from WO 2006/067214. It again follows from WO 2005/111282 to add at least a second polyolefin to a first polypropylene, which is described there as elastomer or plastic reactor grade polypropylene, having at least 3 wt % of a polyethylene. The use of a blend from a first propylene and a second propylene again follows from US 2005/0165173 A1. The first propylene should preferably be a copolymer, in which an ethylene or other olefin is used for this. If the blend is to be used to produce nonwoven materials, it is proposed in the documents to add an additional polymer to the blend, which is miscible or nonmiscible. This can be a polyethylene. For bicomponent fibers material combinations from polypropylene and polyethylene or thermoplastic-elastomer polyolefins and polypropylene are also known, for example, from US 2006/0084342.

The task of the present invention is to devise a cost-effective nonwoven with nevertheless improved properties relative to ordinary polypropylene nonwovens and their fibers without change in other properties, especially tensile strength.

This task is solved with use of a polypropylene blend with the features of Claim 1, with a polypropylene blend with the features of Claim 5, with a spunbond nonwoven with the features of Claim 15 and a method with the features of Claim 22. Additional advantageous embodiments and modifications are mentioned in the corresponding dependent claims.

A polypropylene blend for use in the production of a spunbond nonwoven with increased elongation property is proposed in which a polypropylene blend has essentially a first homopolypropylene and a second homopolypropylene, in which an MFI of the first homopolypropylene is greater than an MFI of the second homopolypropylene, the second homopolypropylene having a weight fraction of polypropylene blend of at least 3 wt % to a maximum 25 wt %, preferably a maximum 23 wt %, in which the first homopolypropylene essentially makes up the remaining weight fraction of the polypropylene blend, the second homopolypropylene having an MFI between 0.7 and 14 g/10 min (230° C./2.1 kg) according to ISO 1133, and a difference of the MFI of the second homopolypropylene from the MFI of the first homopolypropylene is at least 10 g/10 min, and an upper limit of the MFI of the first homopolypropylene is 55 g/10 min (230° C./2.16 kg) according to ISO 1133.

Preferably such a polypropylene blend is produced in order to produce a spunbond nonwoven fiber with it exclusively from the two homopolypropylenes without limited admixture of another polymer. “Essentially” is to be understood in such a case according to this invention to mean that, in addition to the two homopolypropylenes, at most further additives like functional additives are present but the spunbond nonwoven fiber itself consists of the two homopolypropylenes. Ordinary additives can still be added to the material, for example, additives, spinning enhancers, pigments, UV stability-increasing additives, odor-inhibiting additives, additives that additionally influence the surface properties of the spunbond nonwoven fiber, etc. A preferred embodiment of the polypropylene blend therefore consists exclusively of the two homopolypropylenes, optionally with added substance, like additives, but without adding an additional polymer.

A preferred use of the proposed polypropylene blend consists of core-shell fibers. These can have the polypropylene blend as core material or as shell material. It is preferred that the polymer blend is present exclusively in the core or shell. There is also the possibility that the polypropylene blend is used with a first composition as core material and the polypropylene blend with a second composition is used as a shell material. There is also the possibility that the polypropylene blend is also used in other spunbond nonwoven fibers having different materials distributed over the cross section.

According to one embodiment it is proposed that the spunbond nonwoven has a bicomponent spunbond nonwoven with a core having essentially the first and the second homopolypropylene and a shell from especially polyethylene. According to another embodiment such a spunbond nonwoven consists of the first and second homopolypropylene in the core. Such a bicomponent material is preferably used in a laminate, for example, with a film. The film of the second layer so formed is preferably from polyethylene. The polyethylene film is also preferably microporous. According to another embodiment the film is from a liquid-tight material that permits absorption and desorption of moisture so that vapor permeability of the film and therefore the laminate is ensured. For example, the film material can have polyurethane. The film material can have a homopolymer or a copolymer and also be one- or multilayered.

Depending on the application, different material alone or in combination can be used as materials for a film of a laminate or as film-former for a laminate, for example, in different areas of the spunbond nonwoven and/or laminate. A surface of the spunbond nonwoven can be exposed in different areas to different materials or left free. One embodiment proposes use of one or more acrylic polymers. Because of their hydrophobic effect an improvement of the water repellency can be achieved. Their reaction can also be utilized: during application it often happens that extreme expansion occurs with gel formation if crosslinking of the acrylic acid polymer occurs. For example, this permits particularly intimate joining to an adjacent layer into which the acrylic polymer can penetrate, but at least adheres well. On the other hand, embedding can occur in the acrylic polymer, for example, of loose fiber ends or loops of the spunbond nonwoven so that the strength of the joint is increased.

Polyurethane compounds and/or latex compounds can also be used to form a layer of the laminate. These can be made water vapor-permeable and liquid-impermeable and such a layer can also simultaneously serve as support structure. Use of polyurethane makes it possible to adjust a diffusion-open coating. By means of latex there is the possibility of setting a diffusion-tight coating.

Another embodiment proposes use of one or more polyester compounds. An advantage of a polyester layer is the possibility of being able to have high abrasion strength. High tensile strength of the polyester can also be used. Moreover, high heat resistance, insulation and/or damping properties of polyester can also be used if this is used as laminate with a spunbond nonwoven.

In a heat insulation layer of the laminate an air barrier can be achieved by means of a film, which is nonporous, water-tight but water vapor-permeable.

There is also the possibility of using polyamides, EVA, PVAL and/or PVC polymers in order to create a film layer.

A layer of a laminate or a layer contained in the laminate adjacent to the spunbond nonwoven consisting of the polyolefin blend can be a film, a foam, a mesh, a scrim, a woven fabric or other coating. An adjacent layer can also be present on one side and on both sides. An adjacent layer can completely or only partially cover the spunbond nonwoven. The adjacent layer can be joined fully to the spunbond nonwoven or only in areas separated from each other. The adjacent layer can be applied as an independent layer onto the nonwoven, extruded onto it or the spunbond nonwoven can be extruded onto the layer. The adjacent layer can be foamed, sprayed or otherwise applied. Such a laminate can have one or more layers, identical or also different layers. The spunbond nonwoven can form an outer layer of the laminate. The spunbond nonwoven can also be embedded between two or more layers.

According to another embodiment it is proposed that a laminate has a nonwoven layer with a polymer having at least one of the following members of the group including PO, PET, biodegradable polymers, PP, PE, copolymers, antimicrobial additive, hydrophilic additive, phosphorescing additive, fluorescing additive, antistatic additive and dirt repellant additive.

One type of nonwoven or also different types of nonwoven can be used in one laminate. According to one embodiment, for example, carded nonwoven, an SMS material, a film-nonwoven laminate, an air-laid material, a spun-lace material, a melt-blown material, an elastic nonwoven, a biko material and/or a nonwoven can be used whose fibers or filaments has specific geometries, for example, trilobal or other geometries, especially geometries not rounded in cross section. The term bicomponent or multicomponent also pertains to the presence of polymer phases in discrete structured segments in contrast to blends where the domain tends to disperse randomly or in an unstructured fashion. The polymer components can be arranged in each configuration, containing shell-core, side-to-side, segmented pie piece, island-in-the-sea or equipped multilobal geometries as nonwoven fiber cross sections.

It is also preferred that the spunbond nonwoven fiber consists of a proposed polymer blend.

The polypropylene blend according to a modification can have the second homopolypropylene in a range of MFI between preferably 1.8 and 12 g/10 min, especially between 2.3 and 5 g/10 min.

A modification proposes that the first homopolypropylene lies in an MFI range between 16 and 45 g/10 min, preferably between 22 and 38 g/10 min.

According to an embodiment it is proposed, for example, that the second homopolypropylene has an MFI range between 2.3 and 3.5 MFI and an MFI range between 24 and 38 g/10 min for the first homopolypropylene.

For example, it is proposed that the second homopolypropylene has a weight fraction of the polypropylene blend of preferably 5 wt % to 18 wt %, especially 8 wt % to 15 wt %.

According to another embodiment it is proposed that the second homopolypropylene preferably has an MFI in a range between 10 and 15 g/10 min. The first homopolymer preferably has an MFI in the range between 22 and 55 g/10 min, especially 25 g/10 min to 35 g/10 min.

According to one embodiment a commercially available homopolypropylene under the name H502-25RG can be used as first homopolypropylene of the polypropylene blend. This has an MFI of 25 g/10 min according to ISO 1133 with a density of 0.9 g/cm³. Another commercially available homopolypropylene has an MFI of 27 g/10 min according to ISO 1133 with a melting point between 161 and 165° C. This is also usable as first homopolymer. For example, a homopolypropylene marketed by Borealis under the name HG455FB can also be used. Another homopolypropylene also has an MFI of 25 g/10 min, in which this is available from Basell under the name Moplen HP560R.

A homopolypropylene that would otherwise be usable explicitly for the spunbond nonwoven area or fiber area can be used as second homopolypropylene. For example, a homopolypropylene having an MFI of 3.4 g/10 min according to ISO 1133 can be used. This can be a material under the name Moplen HP456J from Basell. Another material, which can be added as second homopolypropylene with a weight fraction between 5 wt % and 25 wt % preferably to 23 wt % has an MFI of 12 g/10 min. For example, this can be a polymer under the name Moplen HP500N. There is also the possibility that another second homopolypropylene with an MFI of 12 g/10 min is used, which is also not otherwise used in spunbond nonwoven production, but comes from injection molding. For example, this can be a polymer from Dow available under the name H779-12. Another second homopolypropylene has an MFI of 0.7 and is available from Basell under the name HP501D.

The proposed polypropylene blend therefore permits homopolypropylenes to be used with each other, in which only one or none of them is normally used for the spunbond nonwoven field. Particularly with respect to the second homopolypropylene there is the possibility of making homopolypropylenes common from other areas, like injection molding useful for application in spunbond nonwoven applications. For example, it can be proposed that a homopolypropylene exclusively usable for nonwoven production is partially replaced by a second homopolypropylene which would not be suited by itself for spunbond nonwoven production.

Another embodiment proposed is that the difference of MFI between the first and second homopolypropylenes is not greater than 30 g/10 min. Another embodiment proposes that the difference is not greater than 15 and preferably lies in the range between 11 and 13 g/10 min.

It is also proposed that a spunbond nonwoven weight of 8 g/m² to 30 g/m² is preferably produced. Especially in lightweight nonwovens in a weight range from 10 g/m² to 15 g/m² an increased elongation property in the nonwoven can be achieved.

Another embodiment proposes that the elongation of polypropylene nonwoven can also be significantly increased if a fraction of a second homopolypropylene with an MFI of 25 g/10 min is mixed with the first homopolypropylene with an MFI of 25 g/10 min.

According to another embodiment it is proposed that the first homopolypropylene was produced with a Ziegler-Natta catalyst in the polypropylene blend, whereas the second homopolypropylene was produced with a metallocene catalyst. Another embodiment proposes that the first homopolypropylene was produced with a metallocene catalyst whereas the second homopolypropylene was produced with a Ziegler-Natta catalyst. Another preferred embodiment proposes that the first and second homopolypropylene each were proposed by means of Ziegler-Natta catalyst. It is therefore possible that, in addition to spinning behavior, which is dictated by the melt flow index in the form of MFI and by molecular weight distribution MWD, an additional effect can be achieved on the properties of the nonwoven or the nonwoven fiber by appropriate adjustment of the polypropylene blend. It is known that a narrowing of the molecular weight distribution offers an improvement in spinning. If the MFI value is increased, an improvement in spinning is often also obtained. It has now turned out that an increase in tensile strength is obtained during choice of a narrow molecular weight distribution, but otherwise stretchability is reduced. By deliberate use of catalyst the property of the polypropylene obtained from it can be deliberately used in order to achieve nonwoven fibers or nonwoven materials with tailor-made elongation and tensile strength.

In addition to use of metallocene and Ziegler-Natta catalysts to produce the homopolymers, other catalysts can also be used. These include half-sandwich amido catalysts, as follows from EP 0 416 815 A1 or EP 0 420 436 A1, as well as diimine complexes, as follow, for example, from WO 96/23010 or WO 98/30612, and which are referred to in the context of this disclosure to this extent.

For example, it is proposed that the first homopolypropylene has an average molecular weight M_{W} lying between 180,000 and 340,000 g/mol. It is also preferred that a molecular weight distribution MWD of the first homopolypropylene is between M_{W}/Mₙ=1.9 and M_{W}/Mₙ=3.7. Another embodiment proposes that the second homopolypropylene has an average molecular weight M_{W} lying between 300,000 and 500,000 mol. Another embodiment proposes that a molecular weight distribution MWD of the second homopolypropylene lies between M_{W}/Mₙ=3.1 and M_{W}/Mₙ=4.8.

Especially from these values for average molecular weight of the first or second homopolypropylene or for the molecular weight distribution of the first or second homopolypropylene different polypropylene blends can be produced in which at least one of the stated ranges, preferably two and especially all four ranges are fulfilled.

For example, if a metallocene catalyst is used in order to produce a homopolypropylene of the polypropylene blend, this preferably has an MFI between 0.7 and 14 g/10 min (230° C./2.16 kg) according to ISO 1133, especially between 2 and 14 g/10 min, and a molecular weight distribution MWD between M_{W}/Mₙ=1.9 and M_{W}/Mₙ=2.5.

According to another idea of the invention a spunbond nonwoven is produced with a polypropylene blend as presented above. According to one embodiment the elongation properties at least in the CD direction, preferably in the CD and MD direction are set greater in comparison with an identical spunbond nonwoven during use of homopolypropylene instead of the polypropylene blend in which the homopolypropylene has an MFI that is obtained by calculation as the mathematical mean from the MFI of the first and second homopolypropylene with consideration of the corresponding weight fractions. This is calculated as follows:

MFI_{comparison}=MFI₁×wt %+MFI₂×wt %.

Preferably the spunbond nonwoven is produced fully from the polypropylene blend in which corresponding additives, spinning enhancers, antioxidants as stated above can be contained, but additional polymers, on the other hand, are dispensed with.

Spunbond nonwoven can have continuous fibers like staple fibers. The spunbond nonwoven method can be carried out with a device as follows from US 2001/0004574 A1 or WO 96/16216 or U.S. Pat. No. 6,207,602. Spunbond nonwoven methods can be used as follow from U.S. Pat. No. 3,692,618, from U.S. Pat. No. 5,032,329, WO 03/038174 or also WO 02/063087. Corresponding devices can also be used to produce bicomponent nonwovens or multicomponent nonwovens, as belonged to the prior art. It is preferred if the spunbond nonwoven is a component of a laminate. The laminate can be two- or multilayers. With reference to the method, devices, possible laminates and other details, the above documents are fully referred to in the context of the disclosure.

For example, the spunbond nonwoven that has the polypropylene blend is contained at least in one layer of the laminate. For example, this can be a single spunbond nonwoven layer. Another layer can be a film. However, there is also the possibility that another layer is another spunbond nonwoven, for example, a nonwoven produced according to a melt-blown method. In particular, the laminate can be an SMS, an FS or an SFS, with S standing for spunbond nonwoven, F for film and M for melt-blown.

If a film is used this is preferably a microporous film. However, an air-tight film can also be used. One application proposes that the laminate is a component of a back sheet of a hygienic product. The hygiene product can be a diaper, a tampon or other. Preferably stretchability of the employed nonwoven is adjusted to the stretchability of the film. The nonwoven weight of a laminate preferably has a nonwoven weight between 10 g/m² and 13 g/m² in which the spunbond nonwoven, preferably the entire laminate is stretched and forms an outer layer of a hygiene product.

The nonwoven, film or the laminate can additionally be finished with hydrophilic agents, with UV stabilizers, γ-stabilizers, flame retardants and/or dyes, especially pigments. A spunbond nonwoven and especially a laminate can be used for a wide variety of application. Preferably the laminate is sterilizable. In addition to use in safety clothing, especially protective clothing, the material can also be used in medical applications, for example, in coverings, bandage material and OR clothing. The material is especially virus-tight. Test methods and also values are apparent from US 2003/124324, which is referred to in the context of this disclosure.

A preferred application of the spunbond nonwoven or the laminate concerns the use in protective clothing. For example, an entire protective clothing can be produced from the laminate. Only part of the protective clothing can also have the laminate. The laminate itself can be bonded with an additional layer, especially with a film layer. Particularly preferred is the use in industrial protective according to Guideline 89/686/EWG category 3 for use as chemical protective clothing according to type 3, 4, 5 or 6. It is preferably proposed for this purpose that the laminate fulfills the test features prescribed for this protective clothing. With reference to these requirements the corresponding test classifications prEN 1511, prEN 1512 or EN 466 and EN 465 for types 3 and 4 for single use or multiple use are referred to. The requirements for type 5 follow from prEN ISO 13982-1:2000-11. The requirements for type 6 follow from prEN 13034:1997-09. These classifications are referred to in this disclosure as features of the employed laminates.

The laminate can also be finished accordingly for different other applications. The finish can occur, for example, by means of an additive. There is also the possibility that a surface application occurs. This can occur, for example, via a spray device, via rolls, wet pick-up or other application devices. There is also the possibility that the laminate is subjected to corona treatment. This can occur, for example, for adjustment of special properties of the laminate. Possible finishes of the laminate, especially by use of additives, include antistatic agents, antimicrobial finishes, UV-resistant finishes, flame protection, alcohol repellency, especially up to 90% alcohol and others. A wide variety of finishes and additives can be used for this purpose. Addition of additives can occur only in one layer but also in at least two layers or all layers of the laminate. For example, a spunbond nonwoven can have a different finish than the film and vice-versa. This is especially true for the aforementioned finishes.

According to another embodiment the spunbond nonwoven or the laminate is used in the packaging field. For example the laminate is sterilized before and/or after, i.e., before and/or after the packing process. The laminate, preferably the entire package is sterilizable. There is also the possibility that the package has several layers in which only part of these layers are sterilizable. For example, one sterilized laminate is arranged on the interior of the package whereas an exterior of the package is not sterilized or not sterilizable.

Other example applications follow from the following documents which are referred to in the context of the disclosure. The proposed polypropylene blend can be used instead of the nonelastic materials that are apparent from the documents.

It is known from US 2003/00 50 589 A1 to use an elastic element to produce and enclosure, for example, for a finger. A base material that would typically be a nonwoven is used for the enclosure. In addition, the base material can also have different other materials, like elastomer components. Different laminates like elastic laminates and film laminates are understood by this in particular. For example, propylene elastic laminates are so-called stretch bonded or neck-bonded laminates. The corresponding definitions of these two materials prescribe that an elastic material is bonded to a nonelastic material. The latter is now a spunbond nonwoven from the proposed polymer blend instead of the described materials.

A stretching apparatus follows from U.S. Pat. No. 6,368,444, by means of which films, nonwovens or laminates are to be simultaneously stretched in the CD and MD direction. The device should be suitable for stretching especially films filled with fillers. Elastomer nonwovens should also be stretchable with the device. In laminates the so-called neck-stretch laminates are stretched in which one layer consists of an elastic material and the other layer of a nonelastic material. Here again the nonelastic material is now produced with the proposed polypropylene blend.

It is known from WO 99/55 942 A1 to extrude a polymer provided with filler material on a staple fiber, which is unstretchable. One fiber of the staple fiber should homogeneous or nonhomogeneous phase mixtures within the fiber through different polypropylene and polyethylene materials in order to obtain a strength for stretching in the CD. Instead of a mixture of polypropylene and polyethylene, the proposed polymer blend is now used to produce the spun fibers.

Another embodiment proposes that the spunbond nonwoven has an at least 20% higher elongation in the CD direction preferably in the CD direction and in the MD direction relative to a second spunbond nonwoven, which is produced essentially exclusively from the first homopolypropylene and is otherwise identical to the spunbond nonwoven of higher elongation.

According to another embodiment it is proposed that elongation in the MD direction remains roughly unchanged relative to a second spunbond nonwoven produced essentially exclusively from the first homopolypropylene and otherwise is identical to the spunbond nonwoven of higher elongation. Preferably the second spunbond nonwoven consists of a first homopolypropylene. The elongation in the CD direction of the spunbond nonwoven produced with the produced polypropylene mixture, on the other hand, is at least 15%, preferably even 25% higher relative to the elongation in the CD direction.

Another advantageous use of the proposed polypropylene blend is obtained in the production of spunbond nonwoven with increased elongation property. In this case a spunbond nonwoven device can be used as is known, for example, as a so-called Reicofil 3 system. There is also the possibility of using other Reicofil technologies like Reicofil 1, 2 or 4 or also Reicofil Biko. The first and the second homopolypropylene can be fed separately to an extruder device and the polypropylene blend produced from it in the extruder device. It is not necessary that a batch be prepared, which is fed to the extruder. Instead the extruder itself can be used to carry out mixing of the first and second homopolypropylene. For example, both homopolymers are metered into the same hopper for this purpose. In addition, an advantage is obtained by using the first and second homopolypropylene in that they are miscible with each other without requiring another additive in order to be able to achieve miscibility of the two materials at all. It is preferred that the first and the second homopolypropylene be fed directly to the same extruder and mixed there. For example, according to one embodiment it is proposed that a single-screw extruder be used.

Another advantageous use of the polypropylene blend is obtained as follows: a spunbond nonwoven device can be stably operated in at least one area with a low temperature during use of the polymer mixture in comparison with use of a homopolypropylene having an MFI that is obtained by calculation as the mathematical average from the MFI of the first and the second homopolypropylene with consideration of the corresponding weight fractions. The corresponding calculation formula is given above. An area can be a section of a heating zone in a screw extruder. However, temperature control of the spin pack can also be involved here. It turned out that by using the two homopolypropylenes a lower energy demand is set relative to use of a comparable individual homopolypropylene with the average MFI value. It also turned out that an increase in extrusion pressure in the spin pack occurs if a percentage of the second homopolypropylene in the polymer blend is increased.

An effect of the two homopolypropylenes is obtained from tests which are enclosed as examples.

TABLE 1

| | die temp | | | | | |
|---|---|---|---|---|---|---|
| | 235° C. | 235° C. | 245° C. | 245° C. | 260° C. | 260° C. |
| | throughput | | | | | |
| | 0.4 g/min | 0.66 g/min | 0.4 g/min | 0.66 g/min | 0.4 g/min | 0.66/g min |
| ratio MFR 25/12 | hole | hole | hole | hole | hole | hole |
| | spinnability | | | | | |
| 100/0 | nt | nt | OK | OK | OK | OK |
| 0/100 | nt | nt | nt | nt | OK | OK |

spin pack pressure (bar)

| | | | | |
|---|---|---|---|---|
| 100/0 | 20 | 24 | 19 | 22 |
| 0/100 | | | 24 | 28 |

extruder power (W)

| | | | | |
|---|---|---|---|---|
| 100/0 | 352 | 690 | 344 | 728 |
| 0/100 | | | 360 | 750 |

The test results for spinnability for the pressure in the spin pack, shown as spin pack pressure in bar and the extruder power shown as extruder power (W) for the two different homopolypropylenes with an MFI of 25 and 12 at different temperatures in the spinning plate area and different hole throughputs, stated as throughput are apparent from Table 1. It can be deduced from this that at a lower MFI a higher pressure but also a higher temperature become necessary. At lower temperatures and lower pressure, on the other hand, the homopolypropylene with lower MFI is not spinnable. With the polymer blend as proposed, however, the possibility is obtained that spinning of the homopolypropylene with lower MFI is made possible by corresponding addition of a second homopolypropylene and at the same time the nonwoven acquires increased elongation relative to a comparative nonwoven as explained above.

The following examples show a section of different tests by means of which the invention can be further explained.

Spunbond nonwovens with different basis weight were produced by melt spinning in such a way that mixtures of homopolypropylenes with different melt flow rate (MFI) were used to produce these spunbond nonwovens. The employed raw materials are apparent from Table 2.

Production of spunbond nonwovens occurred on a so-called Reicofil 3 spunbond nonwoven pilot unit. Only the composition of the mixture but not the chosen process conditions were changed. Additive or color concentrates (master batches) were not added to these blends. However, this can occur fully.

The most important process conditions as well as the properties of the produced spunbond nonwovens with different basis weight are summarized in Tables 2 to 6.

TABLE 2
Employed PP types.

| | | MFI¹⁾ | |
|---|---|---|---|
| Type | Manufacturer | (dg/10 min) | MWD²⁾ |
| Moplen HP560R | Basell | 25 | Very narrow |
| Moplen HP456J | Basell | 3.4 | — |
| H779-12 | Dow | 12 | — |

¹⁾Melt flow rate taken from the technical data sheet
²⁾Molecular weight distribution, information according to technical data sheet

TABLE 3
Spunbond nonwovens with a basis weight of 10 g/m² produced from blends of
PP with MFI 25 and a second PP with MFI 12.

| | | Sample | Sample | Sample | Sample | Sample |
|---|---|---|---|---|---|---|
| | | 10 A-1 | 10 A-2 | 10 A-3 | 10 A-4 | 10 A-5 |
| Composition | | | | | | |
| Moplen HP560R | | 100 | 95 | 90 | 85 | 80 |
| H779-12 | | 0 | 5 | 10 | 15 | 20 |
| Spinneret | | | | | | |
| Capillaries per meter | | 5,000 | 5,000 | 5,000 | 5,000 | 5,000 |
| Processing temperature | | | | | | |
| Extruder 1^{st} zone | ° C. | 180 | 180 | 180 | 180 | 180 |
| Extruder head | ° C. | 235 | 235 | 235 | 235 | 235 |
| Spinneret | ° C. | 240 | 240 | 240 | 240 | 240 |
| Throughput | | | | | | |
| g/hole min | | 0.6 | 0.6 | 0.6 | 0.6 | 0.6 |
| Calendar oil temperature | ° C. | 150 | 150 | 150 | 150 | 150 |
| Calendar nip pressure | N/mm | 70 | 70 | 70 | 70 | 70 |
| Web formation | | | | | | |
| Base weight | g/m² | 10 | 10 | 10 | 10 | 10 |
| Barrier properties | | | | | | |
| Air permeability | 1/m² s | 10.264 | 9.870 | 10.521 | 10.054 | 10.165 |
| Mechanical properties | | | | | | |
| F max MD | N/5 mm | 19.4 | 22.3 | 20.9 | 21.1 | 19.2 |
| F max CD | N/5 mm | 10.9 | 12.2 | 11.9 | 12.7 | 12.6 |
| Elongation MD | % | 51.9 | 66.9 | 62.6 | 66.5 | 63.0 |
| Elongation CD | % | 82.5 | 76.3 | 75.0 | 78.7 | 79.4 |

For example, in this 10 g/m² spunbond nonwoven an elongation increases by continuous addition of a second homopolypropylene in the MD and CD direction. At a nonwoven weight of 10 g/m² or higher, for example, up to 25 g/m² and an MFI of the second homopolypropylene amounting to between 1.7 and 4.5 g/10 min with an MFI of the first homopolypropylene of at least 20 g/10 min, preferably between 25 g/10 min and 45 g/10 min addition of the second homopolypropylene in a range from 3 wt % to 12 wt % is preferred, especially less than 10 wt %.

TABLE 4
Spunbond nonwovens with a basis weight of 14 g/m² produced from blends of a
PP with MFI 25 and a second PP with MFI 12.

| | | Sample | Sample | Sample | Sample | Sample |
|---|---|---|---|---|---|---|
| | | 14 A-1 | 14 A-2 | 14 A-3 | 14 A-4 | 14 A-5 |
| Composition | | | | | | |
| Moplen HP560R | | 100 | 95 | 90 | 85 | 80 |
| H779-12 | | 0 | 5 | 10 | 15 | 20 |
| Spinneret | | | | | | |
| Capillaries per meter | | 5,000 | 5,000 | 5,000 | 5,000 | 5,000 |
| Processing temperature | | | | | | |
| Extruder 1^{st} zone | ° C. | 180 | 180 | 180 | 180 | 180 |
| Extruder head | ° C. | 235 | 235 | 235 | 235 | 235 |
| Spinneret | ° C. | 240 | 240 | 240 | 240 | 240 |
| Throughput | | | | | | |
| g/hole min | | 0.6 | 0.6 | 0.6 | 0.6 | 0.6 |
| Calendar oil temperature | ° C. | 150 | 150 | 150 | 150 | 150 |
| Calendar nip pressure | N/mm | 70 | 70 | 70 | 70 | 70 |
| Web formation | | | | | | |
| Base weight | g/m² | 14 | 14 | 14 | 14 | 14 |
| Barrier properties | | | | | | |
| Air permeability | 1/m² s | 7.575 | 7.595 | 8.020 | 7.608 | 7.815 |
| Mechanical properties | | | | | | |
| F max MD | N/5 mm | 32.4 | 32.5 | 31.1 | 29.9 | 31.4 |
| F max CD | N/5 mm | 18.8 | 18.6 | 19.3 | 20.4 | 18.6 |
| Elongation MD | % | 61.9 | 70.9 | 72.4 | 70.6 | 83.1 |
| Elongation CD | % | 68.0 | 73.1 | 78.0 | 91.1 | 83.5 |

At a nonwoven basis weight of 10 g/m² or high, for example, up to 25 g/m² and an MFI of the second homopolypropylene lying between 10 and 14 g/10 min, with an MFI of the first homopolypropylene of at least 20 g/10 min, preferably between 25 g/10 min and 45 g/10 min addition of the second homopolypropylene is preferred in the range of 8 wt % to 25 wt %, especially more than 10 wt %.

TABLE 5
Spunbond nonwovens with a basis weight of 17 g/m² produced from blends of
PP with MFI 25 and a second PP with MFI 12.

| | | Sample | Sample | Sample | Sample | Sample |
|---|---|---|---|---|---|---|
| | | 17 A-1 | 17 A-2 | 17 A-3 | 17 A-4 | 17 A-5 |
| Composition | | | | | | |
| Moplen HP560R | | 100 | 95 | 90 | 85 | 80 |
| H779-12 | | 0 | 5 | 10 | 15 | 20 |
| Spinneret | | | | | | |
| Capillaries per meter | | 5,000 | 5,000 | 5,000 | 5,000 | 5,000 |
| Processing temperature | | | | | | |
| Extruder 1^{st} zone | ° C. | 180 | 180 | 180 | 180 | 180 |
| Extruder head | ° C. | 235 | 235 | 235 | 235 | 235 |
| Spinneret | ° C. | 240 | 240 | 240 | 240 | 240 |
| Throughput | | | | | | |
| g/hole min | | 0.6 | 0.6 | 0.6 | 0.6 | 0.6 |
| Calendar oil temperature | ° C. | 150 | 150 | 150 | 150 | 150 |
| Calendar nip pressure | N/mm | 70 | 70 | 70 | 70 | 70 |
| Web formation | | | | | | |
| Base weight | g/m² | 17 | 17 | 17 | 17 | 17 |
| Barrier properties | | | | | | |
| Air permeability | 1/m² s | 6.640 | 6.293 | 6.600 | 6.467 | 6.311 |
| Mechanical properties | | | | | | |
| F max MD | N/5 mm | 40.2 | 40.8 | 38.8 | 38.9 | 39.4 |
| F max CD | N/5 mm | 24.8 | 25.0 | 25.1 | 24.5 | 26.6 |
| Elongation MD | % | 67.4 | 75.3 | 75.7 | 80.7 | 88.1 |
| Elongation CD | % | 74.6 | 80.8 | 89.1 | 84.1 | 101.3 |

At a nonwoven weight of 10 g/m² higher, for example, up to 25 g/m² and an MFI of the second homopolypropylene lying between 10 and 14 g/10 min with an MFI of the first homopolypropylene of at least 25 g/10 min, preferably between 30 g/10 min and 55 g/10 min, addition of the second homopolypropylene preferably is in a range from 10 wt % to 25 wt %, especially more than 12 wt %.

TABLE 6
Spunbond nonwovens produced from blends of PP with MFI 25 and
a second PP with MFI 3 with a basis weight of 10 or 15 g/m².

| | | Sample | Sample | Sample | Sample |
|---|---|---|---|---|---|
| | | 10 B-1 | 10 B-2 | 15 B-1 | 15 B-2 |
| Composition | | | | | |
| Moplen | | 100 | 96 | 100 | 96 |
| HP560R | | | | | |
| Moplen | | 0 | 4 | 0 | 4 |
| HP456J | | | | | |
| Spinneret | | | | | |
| Capillaries | | 5,000 | 5,000 | 5,000 | 5,000 |
| per meter | | | | | |
| Processing | | | | | |
| temperature | | | | | |
| Extruder 1^{st} | ° C. | 180 | 180 | 180 | 180 |
| zone | | | | | |
| Extruder head | ° C. | 235 | 235 | 235 | 235 |
| Spinneret | ° C. | 240 | 240 | 240 | 240 |
| Throughput | | | | | |
| g/hole min | | 0.6 | 0.6 | 0.6 | 0.6 |
| Calendar oil | ° C. | 150 | 150 | 150 | 150 |
| temperature | | | | | |
| Calendar nip | N/mm | 70 | 70 | 70 | 70 |
| pressure | | | | | |
| Web formation | | | | | |
| Base weight | g/m² | 10 | 10 | 15 | 15 |
| Barrier | | | | | |
| properties | | | | | |
| Air | 1/m² s | 8.916 | 8.934 | 6.145 | 5.885 |
| permeability | | | | | |
| Mechanical | | | | | |
| properties | | | | | |
| F max MD | N/5 mm | 25.1 | 25.2 | 40.2 | 39.4 |
| F max CD | N/5 mm | 11.3 | 12.0 | 21.1 | 23.2 |
| Elongation MD | % | 60.9 | 81.9 | 73.4 | 86.4 |
| Elongation CD | % | 70.1 | 84.8 | 79.5 | 101.4 |

As the measurements show, at a lower MFI of the second homopolypropylene a lower weight percentage is preferably added to the first homopolypropylene in order to obtain an increase in elongation of more than 10%, especially more than 15%.

The effect of the blends according to the invention can be summarized as follows:

TABLE 7

| | MD elongation | CD elongation |
|---|---|---|
| Blend MFI 25 + | 10 g/m²: increase by >20% | 10 g/m²: increase by >20% |
| MFI 12 | essentially over the entire proposed | essentially over the entire proposed |
| | concentration range of MFI 12 | concentration range of MFI 12 |
| | 14 g/m²: increase by >10%, | 14 g/m²: increase by >10%, |
| | even >30% at 20% addition | increasing as a result of addition |
| | 17 g/m²: increase by >10% | 17 g/m²: increase by >10% |
| | increasing as a result of addition | increasing as a result of addition |
| Blend MFI 25 + | 10 g/m²: increase by >30% at | 10 g/m²: increase by >20% at |
| MFI 3 | 4% addition | 4% addition |
| | 15 g/m²: increase by >15% at | 15 g/m²: increase by >20% at |
| | 4% addition | 4% addition |

It follows from this that with reference to each basis weight of the nonwoven the effect of elongation becomes particularly noticeable and this can also be specially adjusted via the composition of the polypropylene blend: with limited nonwoven weights such increases in elongation can be achieved in a higher basis weight. An increase of at least 20% in the MD elongation is preferably achieved, for example, for basis weight from 10 g/m² to 15 g/m². A CD elongation increase of at least 20% is also preferably achieved.

The effect of a proposed polypropylene blend, for example, in a bicomponent fiber in which the proposed polypropylene blends is provided in the core, is apparent from the following Table 7.

TABLE 8

| | Kern: Standard-PP + MFR | Kern: Standard-PP + MFR |
|---|---|---|
| | 12; Mantel: PE | 12; Mantel: PE |
| | Kern/Mantel-Verh\check{a}ltnis: | Kern/Mantel-Verh\check{a}ltnis: |
| | 50/50 | 70/30 |
| Composition | | |

| | | | | | | | | | |
|---|---|---|---|---|---|---|---|---|---|
| Core | Moplen HP560R | 100 | 95 | 90 | 80 | 100 | 95 | 90 | 80 |
| | Moplen HP500N | 0 | 5 | 10 | 20 | 0 | 5 | 10 | 20 |

| | | | | | | | | | |
|---|---|---|---|---|---|---|---|---|---|
| Sheath Aspun 6834 | | 100 | 100 | 100 | 100 | 100 | 100 | 100 | 100 |
| Spinnaret | | | | | | | | | |
| Capillaries per meter | | 5,000 | 5,000 | 5,000 | 5,000 | 5,000 | 5,000 | 5,000 | 5,000 |
| Processing temperatures | | | | | | | | | |
| Extruder 1st zone | ° C. | 180 | 180 | 180 | 180 | 180 | 180 | 180 | 180 |
| Extruder head | ° C. | 230 | 230 | 230 | 230 | 230 | 230 | 230 | 230 |
| Spinnaret | ° C. | 240 | 240 | 240 | 240 | 240 | 240 | 240 | 240 |
| Throughput | | | | | | | | | |
| g/hole min | | 0.6 | 0.6 | 0.6 | 0.6 | 0.6 | 0.6 | 0.6 | 0.6 |
| Calender oil temperature | ° C. | 136 | 136 | 136 | 136 | 136 | 136 | 136 | 136 |
| Calender nip pressure | N/mm | 70 | 70 | 70 | 70 | 70 | 70 | 70 | 70 |
| Mechanical properties | | | | | | | | | |
| F max MD | N/5 cm | 27.4 | 30.6 | 28.3 | 29.2 | 26.9 | 25.1 | 26.8 | 25.9 |
| F max CD | N/5 cm | 11.3 | 12.2 | 11.0 | 15.3 | 10.6 | 10.1 | 11.5 | 12.7 |

It also surprisingly turned out that despite different mixing of weight fractions of the second homopolypropylene to the first homopolypropylene there is a temperature range during thermocalendering that is less than 10° C. within which the effect of heat bonding is particularly pronounced. This is apparent from the following Table 8.

| | 95% | 90% | 85% | 80% |
|---|---|---|---|---|
| Calander oil | HP560R + | HP560R + | HP560R + | HP560R + |
| temperature | 5% | 10% | 15% | 20% |
| (° C.) | H779-12 | H779-12 | H779-12 | H779-12 |
| | Elongation at peak MD (%) | | | |
| 135 | 38.1 | 45.2 | 40.3 | 43.9 |
| 141 | 60.3 | 63.4 | 76.7 | 71.4 |
| 147 | 78.6 | 80.9 | 93.6 | 92.1 |
| 150 | 70.1 | 79.4 | 88.3 | 91.9 |
| 153 | 72.8 | 78.1 | 81.4 | 79.4 |
| 156 | 67.3 | 74.5 | 67.6 | 82.5 |
| | Elongation at peak CD (%) | | | |
| 135 | 40.0 | 43.7 | 42.6 | 45.7 |
| 141 | 63.5 | 59.4 | 70.3 | 75.8 |
| 147 | 79.7 | 86.8 | 96.0 | 100.2 |
| 150 | 78.7 | 87.0 | 91.9 | 96.9 |
| 153 | 81.0 | 88.8 | 88.7 | 95.1 |
| 156 | 74.7 | 88.2 | 84.3 | 88.9 |

The different calender oil temperatures in ° C. are shown in the first place and the different polypropylene blends from which an individual spunbond nonwoven fiber consists, on the other. It is apparent that at a temperature range between 147° and 153° C. is particularly preferred, since outside this temperature profile a drop in values is found. An exception here is admixing of 20 wt % of the second homopolypropylene. There the temperature value of 156° C. is higher with reference to the MD value relative to that at 143° C. A calendering surface temperature is therefore preferably set that lies in the range between 137° C. and 143° C., i.e., during heat bonding. This reduction relative to the oil temperature is obtained owing to convective heat flows, heat removal to the nonwoven, etc.

### Determination of Properties of Spunbond Nonwovens

Determination of the filament titer occurred by means of a microscope. Conversion of the measured titer (in micrometers) to decitex occurred according to the following formula (density PP=0.91 g/cm³):

$${( \frac{{Titer}_{μ m}}{2} )}^{2} \cdot π \cdot ρ [ \frac{g}{{cm}^{3}} ] \cdot 0.01 = {Titer}_{dtex} [ \frac{g}{{10}^{4} m} ]$$

The basis weight determination of the spunbond nonwovens occurred according to DIN EN 29073-1 on 10×10 cm test specimens.

Measurement of the air permeability with the spunbond nonwovens occurred according to DIN EN ISO 9237. The area of the measurement head was 20 cm², the applied test pressure 200 Pa.

The mechanical properties of spunbond nonwovens were determined according to DIN EN 29073-3. Clamping length: 100 mm, sample width 50 mm, advance 200 mm/min. “Highest tensile force” is the maximum force achieved during passing through the force-elongation curve, “highest tensile force elongation” is the elongation in the force-elongation curve pertaining to the highest tensile force.

Other advantageous embodiments and modifications are apparent from the following drawings. The examples, however, are to be interpreted only as examples and not restrictive. Instead, individual or several features of different figures could be combined with each other to modifications with other features from other figures or from the above description. In the figures

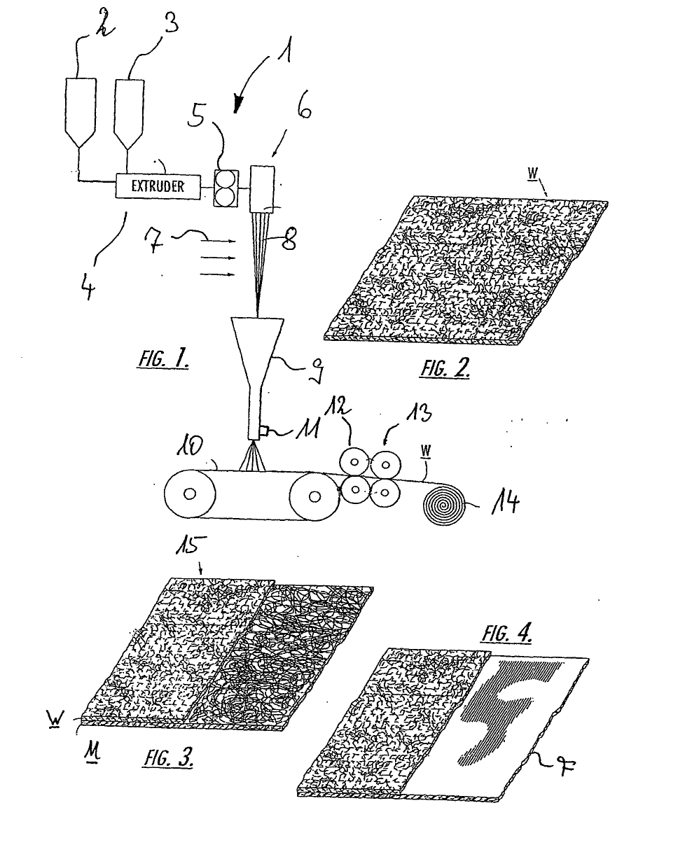

FIG. 1 shows a schematic view of a spunbond nonwoven device in which the polypropylene blend is produced in the extruder,

FIG. 2 shows a spunbond nonwoven produced by means of the polypropylene blend,

FIG. 3 shows a laminate containing a spunbond nonwoven from the proposed polypropylene blend and

FIG. 4 shows a laminate having a spunbond nonwoven from the polypropylene blend joined to a film.

FIG. 1 shows in a schematic embodiment a first spunbond nonwoven device 1. This has a first storage unit 2 for a first homopolypropylene and a second storage unit 3 for a second homopolypropylene. The storage units can be expanded if additional material-like additives are to be added, for example, during use of the first and second homopolypropylene with additional other polymers in bicomponent fibers. The first and second homopolypropylenes are mixed with each other in extruder device 4 in which they are melted there. By the effect of the extruder screw in the interior of the extruder device mixing occurs until a spinning pump 5 is reached. The polypropylene blend so produced is fed by the spinning pump to the spin packs 6 where it emerges and is cooled by a fluid stream 7. The fluid stream 7 preferably uses air for this purpose. The air can be conditioned. This quench can occur on one side or both sides in an open or closed system. A fiber curtain 8 so formed is then fed to a stretching unit 9. The formed nonwoven fibers are laid on a laying belt 10 from the stretching unit 9. The stretching unit 9 can be connected to an electrostatic charging unit 11. A diffuser can also be arranged directly on the stretching unit 9 or adjacent to it in the flow direction of the fibers before the laying belt 10. In this way spreading and therefore improved laying of the nonwoven fibers can occur. The nonwoven fibers are then joined to each other after laying, for example, by a heat-bonding calender 12. So-called stretching of the formed spunbond nonwoven W can then occur. This is shown by a stretching unit 13. The stretching unit 13 can have a ring-rolling unit. In it disks engage opposite calendering rolls and in this way stretch the material. With reference to stretching U.S. Pat. No. 6,042,575 is referred to. This includes among other things in the description a ring-rolled top sheet with reference to another patent, U.S. Pat. No. 4,107,364, in which a ring-rolling process is mentioned in the description and the drawings. Stretching can occur in the CD and MD direction. In addition to or instead of a calendering unit, a stretching frame can also be used. The spunbond nonwoven W is then wound by means of a winding unit 14 and made ready for transport with it.

The depicted spunbond nonwoven device 1 is only an example. One or more additional spinning beams can be integrated in it. These can also be spinning means for production of continuous nonwoven fibers. However, one or more melt-blown beams can also be present. There is also the possibility that the prefabricated material is fed from a unwinding unit, which is further shown, to the spunbonded nonwoven device 1. The prefabricated material can be a nonwoven, a film or also a laminate or different materials. In addition there is the possibility that after the depicted spunbond nonwoven device but before the heat-bonding calender a flowable polymer material is added which can form a film. This can be filled with chalk or another filler. By subsequent heat bonding an additional strength between the at least two layers containing the spunbond nonwoven is created. Subsequent stretching, for example, creates air permeability or gas permeability in the film material. This can become microporous in this way. By the level of stretching the microporosity and therefore the property of the laminate can be set. Support of adhesion preferably occurs by making one or more additional bonds between the first and second layer. A bond between the layers can be made for example by a heat-bonding step, by means of needling, by water jet strengthening or ultrasonic welding. Adhesive fibers can also be used.

FIG. 2 shows a depiction of the spunbond nonwoven W. This has a surface pattern that is made possible by the heat-bonding step and corresponding embossing surfaces. The surface can also be provided with loose fibers, but in which most are bonded to the surrounding fibers by heat bonding. Elongation in the CD or MD direction is increased by the employed polypropylene blend relative to use of an individual homopolymer corresponding to the MFI value.

FIG. 3 shows a laminate 15 with the spunbond nonwoven W already known from FIG. 2. A melt-blown layer M is joined to it. The laminate formed in this way can find use for example in hygienic applications, filter systems or other applications.

FIG. 4 shows a laminate from the film F and the spunbond nonwoven W. The transverse grooves in film F indicate that the film is stretched. Because of this a microporosity of the film is achieved. In addition to the microporous film F, however, another type of film can also be used, for example, a diffusion-open film. This permits wind tightness but at the same time transmission of moisture. Such a laminate can find use in hygienic applications, but especially in construction, for example, roof underlayment or as a wall covering.

1. Use of a polypropylene blend for production of spunbond nonwoven with increased elongation property in which the polypropylene blend essentially has a first homopolypropylene and a second homopolypropylene, an MFI of the first homopolypropylene being greater than an MFI of the second homopolypropylene in which the second homopolypropylene has a weight fraction in the polypropylene blend of at least 3 wt % to maximum 25 wt %, essentially the first homopolypropylene making up the remaining weight fraction of the polypropylene blend, in which the second homopolypropylene has an MFI between 0.7 and 14 g/10 min (230° C./2.16 kg) according to ISO 1133, and a difference of the MFI of the second homopolypropylene from the MFI of the first homopolypropylene is at least 10 g/10 min, and an upper limit of the MFI of the first homopolypropylene amounts to 55 g/10 min (230° C./2.16 kg) according to ISO 1133.

2. Use according to claim 1, characterized by the fact that the second homopolypropylene is used with a weight fraction in the polypropylene blend from 5 wt % to 18 wt %, especially 8 wt % to 15 wt %.

3. Use according to claim 1 or 2, characterized by the fact that the polypropylene blend is used as core material or shell material of a core-shell fiber.

4. Use according to claim 1 or 2, characterized by the fact that only the polypropylene blend is used to produce a spunbond nonwoven fiber.

5. Polypropylene blend for spunbond nonwoven production according to one of the claims 1 to 4, characterized by the fact that the polypropylene blend has essentially a first homopolypropylene and a second homopolypropylene, in which an MFI of the first homopolypropylene is greater than an MFI of the second homopolypropylene, the second homopolypropylene having a weight fraction of polypropylene blend of at least 3 wt % to a maximum 25 wt %, in which the first homopolypropylene essentially makes up the remaining weight fraction of the polypropylene blend, the second homopolypropylene having an MFI between 0.7 and 14 g/10 min (230° C./2.16 kg) according to ISO 1133, and a difference of the MFI of the second homopolypropylene from the MFI of the first homopolypropylene is at least 10 g/10 min (230° C./2.16 kg) according to ISO 1133 and an upper limit of the MFI of the first homopolypropylene is 55 g/10 min (230° C./2.16 kg) according to ISO 1133.

6. Polypropylene blend according to claim 5, characterized by the fact that the second homopolypropylene has a weight fraction in the polypropylene blend from 5 wt % to 18 wt %, especially 8 wt % to 15 wt %.

7. Polypropylene blend according to claim 5 or 6, characterized by the fact that the first homopolypropylene is produced with a Ziegler-Natta catalyst whereas the second homopolypropylene is produced with a metallocene catalyst.

8. Polypropylene blend according to claim 5 or 6, characterized by the fact that the first homopolypropylene is produced with a metallocene catalyst whereas the second homopolypropylene is produced with a Ziegler-Natta catalyst.

9. Polypropylene blend according to one of the claims 5 to 8, characterized by the fact that the first homopolypropylene has an average molecular weight M_{W} between 180,000 and 340,000 g/mol.

10. Polypropylene blend according to one of the claims 5 to 9, characterized by the fact that the molecular weight distribution MWD of the first homopolypropylene is between M_{W}/Mₙ=2.3 and M_{W}/Mₙ=3.7.

11. Polypropylene blend according to one of the claim 5 or 6, characterized by the fact that the first and second homopolypropylene are each produced with a Ziegler-Natta catalyst.

12. Polypropylene blend according to one of the claims 5 to 11, characterized by the fact that the second homopolypropylene has an average molecular weight M_{W} between 300,000 and 500,000 g/mol.

13. Polypropylene blend according to one of the claims 5 to 12, characterized by the fact that the molecular weight distribution MWD of the second homopolypropylene is between M_{W}/Mₙ=3.1 and M_{W}/Mₙ=4.8.

14. Polypropylene blend according to one of the claims 1 to 13, characterized by the fact that the homopolypropylene produced with a metallocene catalyst has an MFI between 0.7 and 14 g/10 min (230° C./2.16 kg) according to ISO 1133, preferably an MFI between 2 and 14 g/10 min and a molecular weight distribution MWD between M_{W}/Mₙ=1.9 and M_{W}/Mₙ=2.5.

15. Spunbond nonwoven with spunbond nonwoven fibers consisting over the entire cross section or consisting in multicomponent fibers in an area of the cross section delimited from another component essentially of a polypropylene blend according to one of the claims 1 to 14.

16. Spunbond nonwoven according to claim 15, characterized by the fact that the elongation properties in the CD direction, preferably in the CD and MD direction of the spunbond nonwoven are greater in comparison to an identical spunbond nonwoven during use of a homopolypropylene instead of the polypropylene mixture, in which the homopolypropylene has an MFI that is obtained by calculation as the mathematical average from the MFI of the first and second homopolypropylene with consideration of the corresponding weight fraction.

17. Spunbond nonwoven according to claim 15 or 16, characterized by the fact that it is a component of a laminate.

18. Spunbond nonwoven according to claim 15, 16 or 17, characterized by the fact that it is a component of a breathable laminate with microporous film.

19. Spunbond nonwoven according to one of the claims 15 to 18, characterized by the fact that it is a component of a back sheet of a hygienic product.

20. Spunbond nonwoven according to one of the claims 15 to 19, characterized by the fact that it has a nonwoven weight between 10 g/m² and 15 g/m², is stretched and forms an outer layer of a diaper.

21. Spunbond nonwoven according to one of the claims 15 to 20, characterized by the fact that spunbond nonwoven has an at least 20% higher elongation in the CD and in the MD direction relative to a second spunbond nonwoven that is produced from essentially exclusively the first homopolypropylene and is otherwise identical to the spunbond nonwoven of higher elongation.

22. Method of production of a spunbond nonwoven with increased elongation property in which a polypropylene blend according to one of the claims 1 to 14 is used to produce a spunbond nonwoven essentially consisting of it.

23. Method according to claim 22, characterized by the fact that the first and second homopolypropylene are each supplied separately to an extruder device and the polypropylene blend is produced from them in the extruder device.

24. Method according to claim 22 or 23, characterized by the fact that the first and the second homopolypropylene are each fed directly to the same extruder and mixed there.

25. Method according to claim 22, 23 or 24 characterized by the fact that a single-screw extruder is used.

26. Method according to one of the claims 22 to 25, characterized by the fact that a spunbond nonwoven production device during use of the polymer blend is stably operated in at least one area with a lower temperature in comparison to use of a homopolypropylene having an MFI that is obtained by calculation as a mathematical average from the MFI of the first and the second homopolypropylene with consideration of the corresponding weight fractions.

27. Method according to one of the preceding claims, characterized by the fact that an increase in extrusion pressure occurs in the spin pack when a fraction of the second homopolypropylene in the polymer blend in increased.

28. Method according to one of the preceding claims, characterized by the fact that in a heat-bonding step in at least one heated roll of a roll calender a surface temperature between 136° C. and 143° C. is set.
